(19) United States
(12) Patent Application Publication
(10) Pub. No.: US 2013/0079815 A1
(43) Pub. Date: Mar. 28, 2013

(54) Interlocking Sutures

(71) Applicant: Surgimatix, Inc., Hillside, IL (US)

(72) Inventors: Jafar Syed Hasan, Oak Brook, IL (US); Wai Ngai Chin, Glenview, IL (US); Gary Michael Kobylewski, Hoffman Estates, IL (US)

(73) Assignee: Surgimatix, Inc., Hillside, IL (US)

(21) Appl. No.: 13/629,786

(22) Filed: Sep. 28, 2012

Related U.S. Application Data
(60) Provisional application No. 61/540,076, filed on Sep. 28, 2011.

(51) Int. Cl. A61B 17/04

(52) U.S. Cl. 606/228

(57) ABSTRACT

A surgical suture with improved suture and/or knot security is disclosed. The suture may include an elongated core extending along an axis, and at least one axial array of projections provided on the core. Each projection extends from the core and terminates into an outer edge. The outer edge of the projection may have an axial length greater than that of a remainder of the projection. A method of using the suture to form a surgical stitch is also disclosed.

### CROSS-REFERENCE TO RELATED APPLICATION

This application is based on and claims priority from U.S. Provisional Application Ser. No. 61/540,076, filed on Sep. 28, 2011.

### BACKGROUND

1. Technical Field

This disclosure generally relates to surgical sutures and, more particularly, relates to surgical sutures with interlocking projections to provide improved suture security, knot security and/or other benefits to wound closure or tissue repair.

2. Description of the Related Art

Surgical sutures are widely used in medical procedures to close wounds and surgical incisions, and to repair damaged or severed muscles, vessels, and other body tissues. Generally, a suture is attached at one end to a needle, and the needle is drawn through tissue to form one or more loops holding the tissue together. The suture is subsequently tied off in one or more knots so that the tissue will remain drawn together. With “interrupted” sutures, each placement of the stitch is finished with a tied knot and the remaining ends of the suture are cut. With running sutures, the initial pass of the suture is tied into an anchoring knot before the suture is stitched over along the wound interface. Once this running stitch reaches the end of tissue interface to be fastened, an end knot is tied to secure the entire suture.

Thus, effective tissue fastening or wound closure with sutures involves not only precise suture placement but also secured formation and placement of the knots. Specifically, surgeons must tie sutures in a manner that ensures “knot security” (the ability of a knot to remain securely fastened). In addition to being influenced by the specific manner in which a surgeon ties knots, knot security may also be dependent on the type of suture used. For example, a monofilament suture generally has a smooth outer surface, which increases the risk of loops of the knot slipping out or disentangling, leading to less knot security. On the other hand, braided or stranded sutures have more variations/irregularities at the outer surface that increases the friction of the entangling interaction within the knot, thereby leading to higher knot security. To offset the lower knot security associated with monofilament suture, surgeons often put more “throws” (i.e. loops) in knots when tying monofilament suture in order to prevent the knots from disentangling. For example, it is common to put four, five, or even more throws in a single knot when monofilament sutures are used. On the other hand, surgeons tying braided sutures may use fewer throws to a knot because braided sutures generally have higher knot security due to higher surface friction.

Although sutures are effective for closing wounds, there are a number of issues associated with using conventional sutures. Many of these issues are directly related to using knots to secure the sutures in place. If the knots are not tied properly, defects may arise including knot slippage and re-opening of the wound. Moreover, the use of more “throws” or braided sutures to improve knot security would generally increase the mass of foreign material in the tissue interface, leading to higher risk of inflammation and scarring. Another issue with a conventional suture is the exposure of knots (particularly thick knots formed by several throws) through the surgical site, i.e. the extrusion of sutures through the surgical incisions (also known as “knot spitting”).

To address some of the problems associated with conventional sutures, barbed sutures have been developed to secure tissues. Unlike conventional sutures, barbed sutures have projecting barbs that allow the suture to be used to close wounds, approximate tissue, tighten tissue, and attach prosthetic devices- all without using knots. Fixing conventional sutures with knots requires the knots to be pushed down toward the tissue to assure proper tensioning and fixation of the sutures. The knot then forms a “closed loop” that engages the tissue. In contrast, barbed sutures achieve proper tensioning and fixation by the barbs engaging the tissue path through which the suture passes.

Nevertheless, barbed sutures have their own drawbacks. First, as the security of barbed sutures within a tissue relies entirely on the engagement between the barbs and the tissue, barbed sutures generally have decreased holding strength compared to conventional sutures with secured knots. Moreover, because barbed sutures typically run back and forth across the tissue interface (e.g. wound or incision) to increase the length of the tissue engagement for better security, the tissue interface may exhibit some tension-induced deformation, such as serpentine or sinusoidal configurations, particularly if the back and forth stitching is performed improperly during the surgical procedure.

Thus, there is a need for sutures with improved knot and/or suture security while maintaining its holding strength. In addition, there is a need for sutures that simplify surgical procedures and minimize the likelihood of surgical error. Finally, there is a need for sutures having improved knot/suture security but without increasing the risk of tension-induced deformation around tissue interface.

### SUMMARY OF THE DISCLOSURE

In accordance with one aspect of the present disclosure, a surgical suture is disclosed as including an elongated core extending along an axis, and at least one axial array of projections provided on the core. Each projection extends from the core and terminates into an outer edge. The outer edge of the projection may have an axial length greater than a remainder of the projection.

In accordance with another aspect of the present disclosure, a surgical suture is disclosed as including an elongated core extending along an axis, and at least one axial array of projections extending from the core. Each two adjacent projections define a slot. Each slot may include a radial opening having an axial length smaller than a remainder of the slot.

In accordance with yet another aspect of the present disclosure, a method for forming a surgical stitch is disclosed. The method includes the steps of providing a surgical suture comprising an elongated core extending along an axis and at least one axial array of projections extending from the core, and intersecting the surgical suture to form at least one loop, wherein the intersection is secured by at least one of projection-projection engagement and projection-core engagement. The method may further include the step of tying at least one knot over the loop.

These and other aspects and features of the disclosure will be better understood upon reading the following detailed description in conjunction with the accompanying drawings.

### BRIEF DESCRIPTION OF THE DRAWINGS

For a more complete understanding of the disclosed surgical suture and the use thereof to form surgical stitches, reference should be made to the embodiments illustrated in greater detail in the accompanying drawings, wherein:

FIG. 1 is a perspective view of a surgical suture according to a first embodiment of the present disclosure;

FIG. 2 is an cross-sectional view of the surgical suture in FIG. 1 (taken along 1-1′), particularly illustrating the arrangement of the two arrays of projections around the core;

FIG. 3 is a perspective view of a surgical suture according to a second embodiment of the present disclosure;

FIG. 4 is an cross-sectional view of the surgical suture in FIG. 3 (taken along 3-3′), particularly illustrating the curved projections;

FIG. 5 is a perspective view of a surgical suture according to a third embodiment of the present disclosure;

FIG. 6 is an cross-sectional view of the surgical suture in FIG. 5 (taken along 5-5′), particularly illustrating the angular projections;

FIG. 7 is a perspective view of a surgical suture according to a fourth embodiment of the present disclosure;

FIG. 8 is an cross-sectional view of the surgical suture in FIG. 7 (taken along 7-7′), particularly illustrating particularly illustrating the arrangement of three arrays of projections around the core;

FIG. 9 is a perspective view of a surgical suture according to a fifth embodiment of the present disclosure;

FIG. 10 is an cross-sectional view of the surgical suture in FIG. 9 (taken along 9-9′), particularly illustrating particularly illustrating the arrangement of four arrays of projections around the core;

FIG. 11 is a perspective view of a surgical suture according to a sixth embodiment of the present disclosure;

FIG. 12 is a perspective view of a surgical suture according to a seventh embodiment of the present disclosure;

FIG. 13 is a perspective view of a surgical suture according to an eighth embodiment of the present disclosure;

FIG. 14 is a perspective view of a suture intersection according to another aspect of the present disclosure, particular illustrating projection-core engagement;

FIG. 15 is a graphic illustration of using the disclosed suture to form a regular interrupted stitch;

FIG. 16 is a graphic illustration of using the disclosed suture to form a subcutaneous interrupted stitch;

FIG. 17 is a graphic illustration of using the disclosed suture to form a regular running stitch; and

FIG. 18 is a graphic illustration of using the disclosed suture to form a subcutaneous running stitch.

It should be understood that the drawings are not necessarily to scale and that the disclosed embodiments are sometimes illustrated diagrammatically and in partial views. In certain instances, details which are not necessary for an understanding of the disclosed apparatus or method which render other details difficult to perceive may have been omitted. It should be understood, of course, that this disclosure is not limited to the particular embodiments illustrated herein.

### DETAILED DESCRIPTION OF THE DISCLOSURE

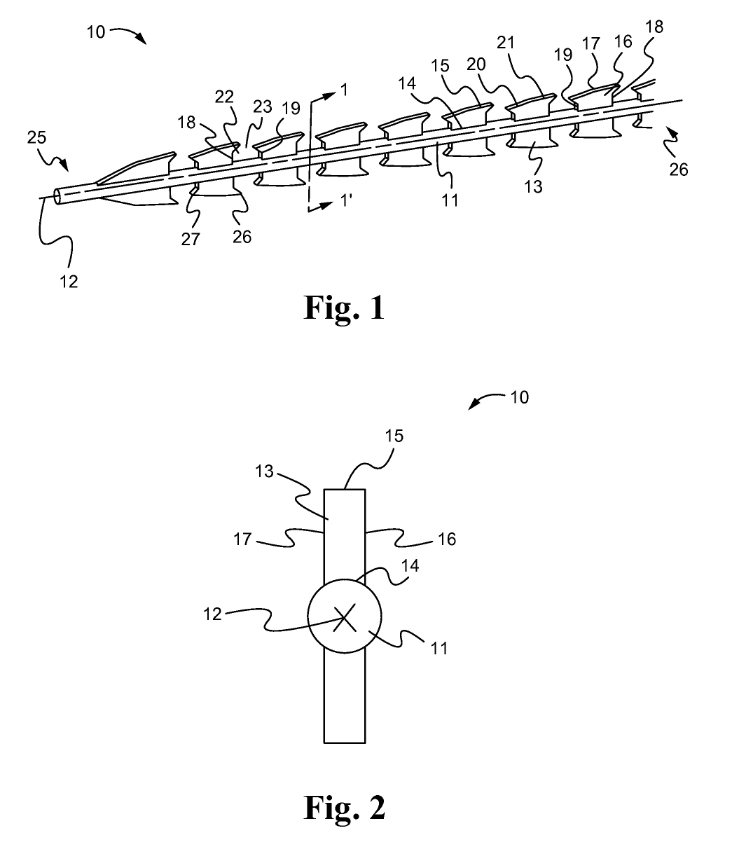

Referring now to the drawings, and with specific reference to FIG. 1, a non-limiting embodiment of a surgical suture 10 according to the present disclosure is illustrated as generally including an elongated core 11 extending along an axis 12 and between a proximal end 24 to a distal end 25. The term “distal” in the present disclosure refers to a direction in which the suture 10 advances into or through a tissue. On the other hand, the term “proximal” in the present disclosure refers to a direction in which the suture 10 retracts out of or through a tissue. The core 11 may be mono-filamentous. The suture 10 further includes at least one axial array of projections 13 provided on the core 11. As illustrated in FIGS. 1-2, the suture 10 may include two arrays of projections 13 extending from the core 11 in opposite directions. Sutures including only one axial array of projections are also within the scope of this disclosure, although not particularly illustrated in the drawings. Moreover, although the core 11 is illustrated as having a circular cross sectional profile, it may take other cross sectional shapes including, but not limited to, oval, triangle, rectangular, square, polygonal (e.g. hexagonal and octagonal), and other suitable shapes in light of the present disclosure.

Still referring to FIGS. 1-2, each projection 13 extends from a base 14 (connected to or integrated with the core 11) to an outer edge 15. The outer edge 15 extends between a proximal end 26 and a distal end 27. In one embodiment, the distal end 27 is positioned closer to the core 11 than the proximal end 26 to reduce premature tissue engagement when the suture 10 is distally advanced within the tissue. The shielding of the distal end 27 by the proximal end 26 may also reduce the likelihood of premature and accidental suture-to-suture engagement while the suture is distally advanced within the tissue. The projection 13 further includes two sidewalls (16, 17) and two end walls (18, 19) extending between the base 14 and the outer edge 15. The two sidewalls (16, 17) may be at least substantially parallel to each other and may have dimensions significantly greater than those of the end walls (18, 19). As a result, the projections 13 are shaped like “fins” and are therefore structurally different from the projections in barbs sutures.

As illustrated in FIG. 1, the outer edge 15 of the projection 13 may have an axial length greater than that of a remainder of the projection 13, including the base 14. The outer edge 15 of the projection may be parallel to the axis 12 in some embodiments and non-parallel to the axis 12 (e.g. curved or angular) in other embodiments. For example, the outer edge 15 in FIG. 1 is angular and includes interconnected sections (20, 21) positioned at different angles relative to the axis 12. Without wishing to be bound by any particular theory, it is contemplated that the specific shape of the projections 13 may allow the suture of the present disclosure to form interlocking intersections to improve suture security and/or knot security. Moreover, the specific shape of the projections 13 may also contribute to suture security through suture-tissue interaction while the interlocking feature allows improved knot security.

Still referring to FIG. 1, each two adjacent projections 13 define a slot 22 that starts from the core and radially terminates at an opening 23. Each slot 22 is defined by the core 11 and the end walls (18, 19) of the adjacent projections 13. According to one aspect of the present disclosure, the opening 23 of each slot 22 may have an axial length smaller than that of a remainder of the opening 22. Without wishing to be bound by any particular theory, it is contemplated that the specific shape of the slots 22 may allow the suture of the present disclosure to form interlocking intersections to improve suture security and/or knot security. Moreover, the specific shape of the slots 22 may also contribute to suture security through suture-tissue interaction while the interlocking feature allows improved knot security.

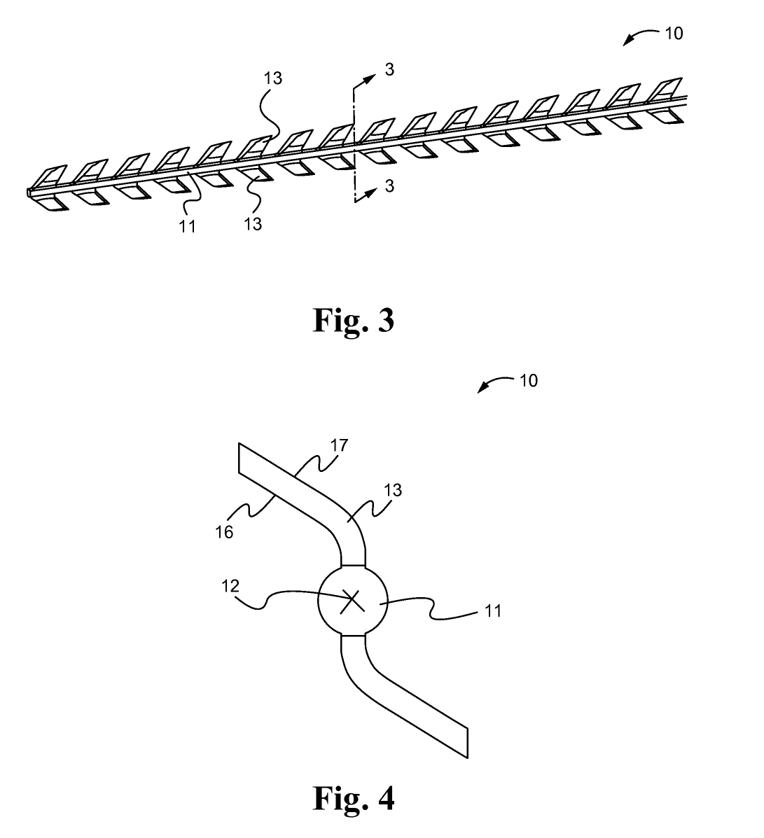

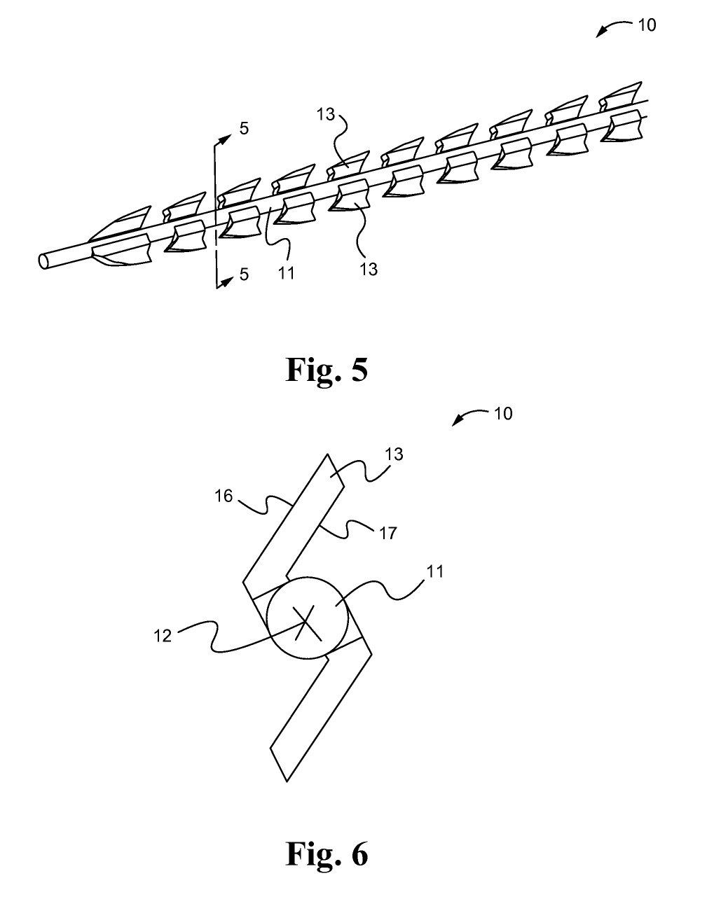

While the projections 13 shown in FIGS. 1-2 are planar, they may be non-planar (e.g. curved or angular) in other embodiment. For example, the projections 13 and their sidewalls (16, 17) may be curved, as shown in FIGS. 3-4; or they may be angular, as shown in FIGS. 5-6. The non-planar projections 13 may be symmetrically oriented around the core 11. Without wishing to be bound by any particular theory, it is contemplated that the curved or angular shape and/or symmetrical orientation of the projections 13 may allow the suture of the present disclosure to form interlocking intersections to improve suture security and/or knot security. Moreover, the curved or angular shape and/or symmetrical orientation of the projections may also contribute to suture security through suture-tissue interaction while the interlocking feature allows improved knot security.

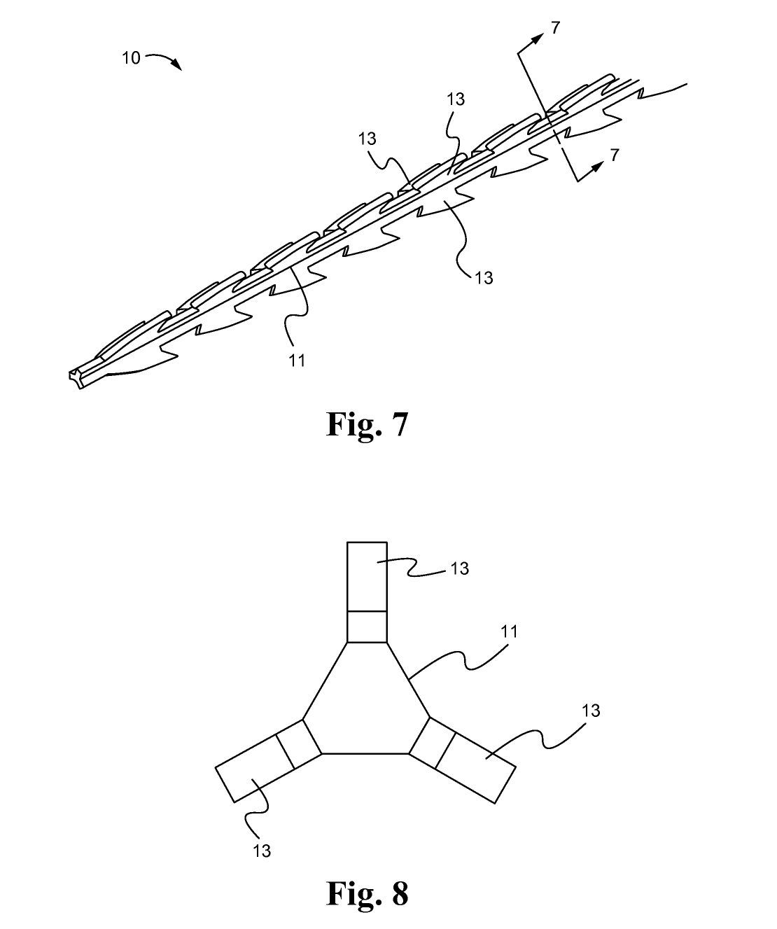

In some embodiments, the suture 10 may include more than two axial arrays of the projections 13 to further enhance suture and/or knot security. As illustrated in FIGS. 7-8, the suture 10 may include three axial arrays of the projections 13 radially extending from the core 11, which may have a hexagonal cross-sectional profile. The three arrays of the projections 13 may be evenly spaced and symmetrically oriented around the core 11 (i.e. intersecting with each other at an angle of)120° . Although the projections 13 shown in FIGS. 7-8 are planar, they may be curved or angular in other embodiments. Moreover, although the outer edges 15 of the projections 13 are angular in FIGS. 7-8, they may be curved or straight in other embodiments.

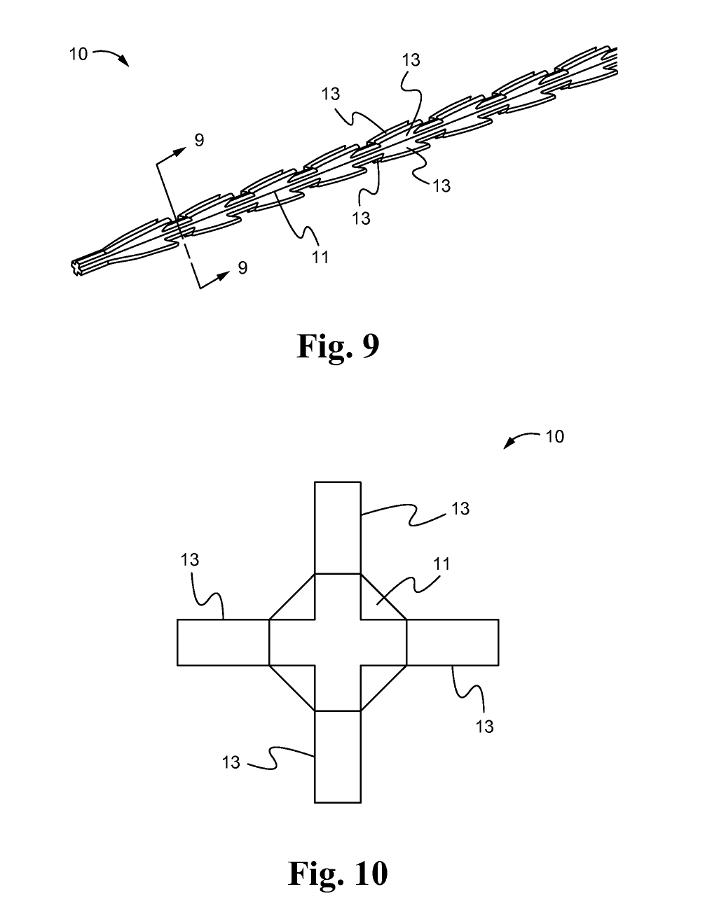

Turning now to FIGS. 9-10, the suture 10 may include four axial arrays of the projections 13 radially extending from the core 11, which may have an octagonal cross-sectional profile. The four arrays of the projections 13 may be evenly spaced and symmetrically oriented around the core 11 (i.e. intersecting with each other at an angle of) 90°. Again, although the projections 13 shown in FIGS. 9-10 are planar, they may be curved or angular in other embodiments. Moreover, although the outer edges 15 of the projections 13 are angular in FIGS. 7-8, they may be curved or straight in other embodiments.

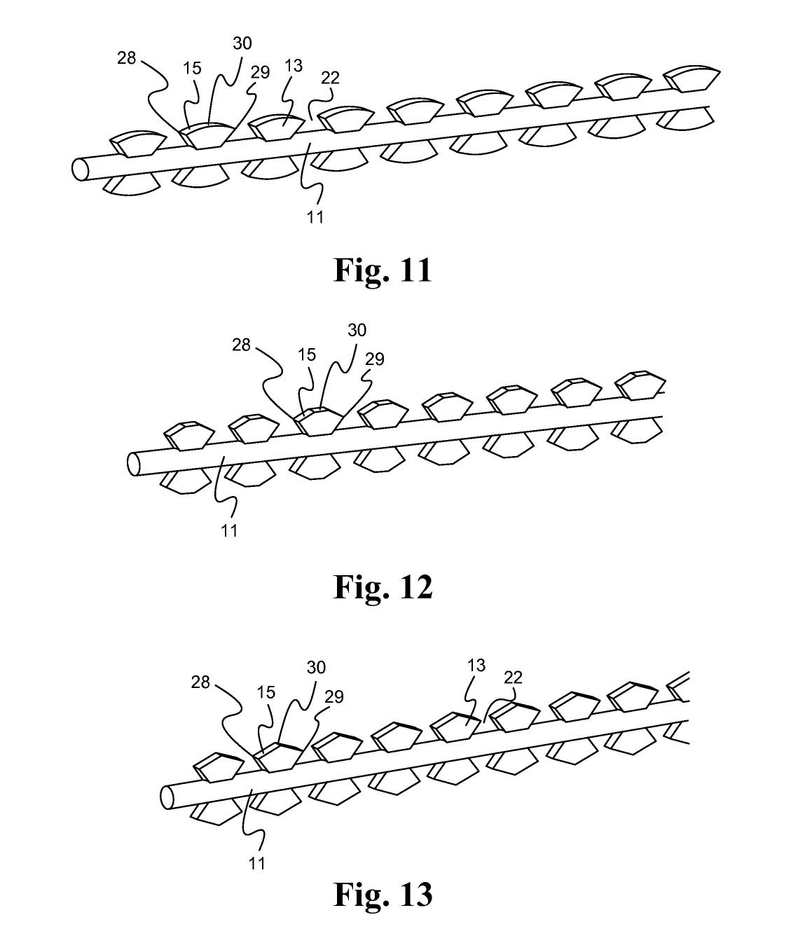

The suture 10 of the present disclosure may also be bi-directional in some embodiments. As illustrated in FIGS. 11-13, the outer edge 15 may include an outer edge 15 having two ends (28, 29) and an apex 30. Again, the outer edge 15 may be curved (FIG. 11) or angular (FIGS. 12-13). In a refinement, the two ends (28, 29) of the outer edge may be equidistant to the center core center core.

Without wishing to be bound by any particular theory, it is contemplated that such a configuration may allow the two ends (28, 29) to be shielded by the apex 30 to reduce tissue resistance and/or tissue damage regardless of the direction of the suture 10. In addition, the shielding of the ends (28, 29) may reduce the likelihood of premature and accidental suture-to-suture engagement while the suture is still being threaded within a tissue. In other word, by shielding the ends (28, 29), the suture 10 may form an interlocking structure only when a projection 13 is properly seated in the slot 22 formed between two adjacent projections 13.

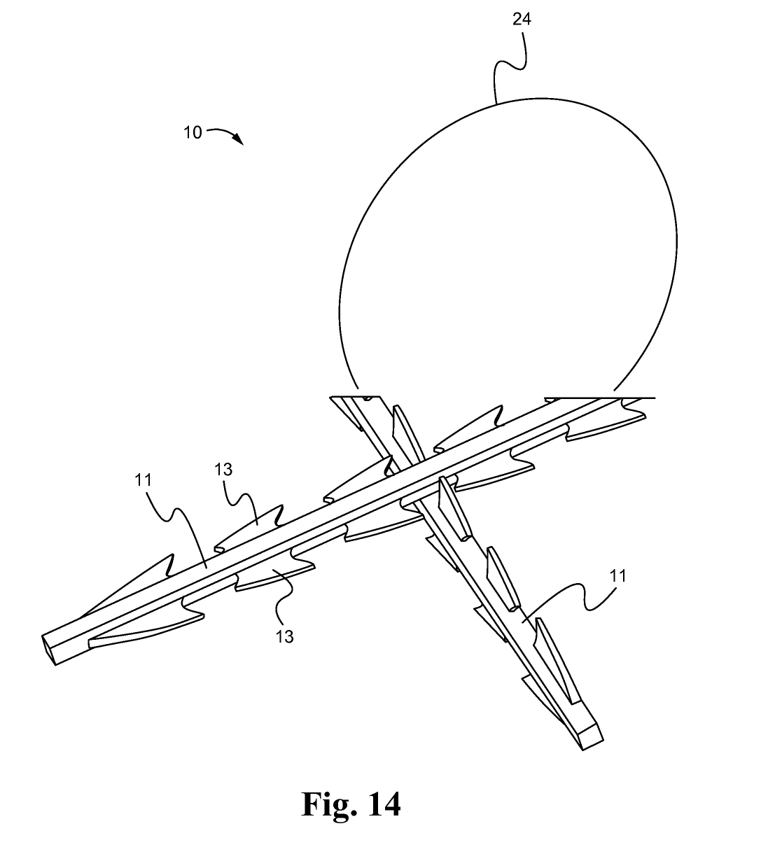

The suture 10 according to the present disclosure may be manufactured through injection molding. The suture 10 may also be manufactured through extrusion-based processes, stamping-based processes, or other suitable manufacturing methods in light of the present disclosure. To use the suture 10 of the present disclosure, a surgeon may insert the suture 10 through a tissue or wound interface and cross or intersect the suture 10 to form one or more loops 24. As generally illustrated in FIG. 14, the intersection of the suture 10 may allow at least one of the projections 13 to engage other projections 13, the core 11, or both. In some embodiments, the engagement between the projection 13 and the core 11 may be enhanced by shaping the core 11 with an edged cross-sectional profile (e.g. triangular, rectangular, square, and polygonal). To further improve the suture security, the surgeon may tie one or more knots (not shown) over the loop 24.

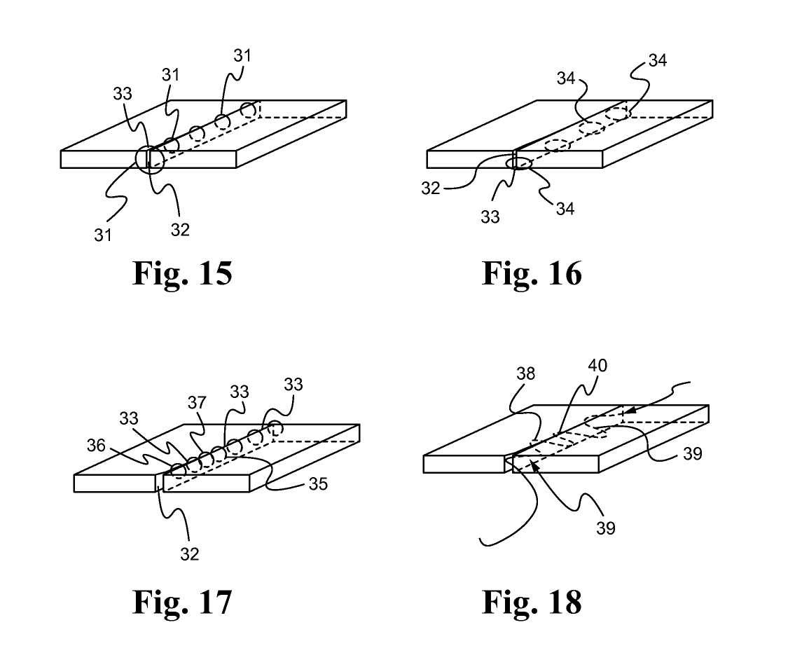

Turning now to FIG. 15, an exemplary interrupted stitch 31 in a tissue or wound interface 32 can be formed by inserting the disclosed suture 10 across the interface 32, intersecting the suture 10 to form an interlocking structure 33, and optionally tying one or more knots over the interlocking structure 33. As illustrated in FIG. 15, a plurality of the interruptive stitches 31 may be formed along the length of the interface 32 to effectively close the wound or tissue interface 32. Unlike traditional interrupted stitches with traditional suture, interrupted stitches 31 may require as little as one loop of intersection for each knot (i.e. as little as one “throw”).

In some procedures, especially when optimal scar appearance is important, it would be desirable to form the stitches beneath the surface of the wound or tissue interface 32. To that end, FIG. 16 illustrates a subcutaneous or deep-dermal interrupted stitch 34 that can be formed by the disclosed suture 10. Specifically, the subcutaneous or deep-dermal interrupted stitch 34 can be formed by inserting the disclosed suture 10 from the undersurface of the wound interface 32, intersecting the suture 10 below the superficial skin surface to form an interlocking structure 33, and optionally tying one or more subcutaneous knots over the interlocking structure 33. As illustrated in FIG. 16, a plurality of the subcutaneous or deep-dermal interrupted stitch 34 may be formed along the length of the interface 32 to effectively close the wound or tissue interface 32. Unlike traditional interrupted stitches with traditional suture, interrupted stitches 34 may require as little as one loop of intersection for each knot (i.e. as little as one “throw”).

In addition to interrupted stitches, the disclosed suture 10 may also be used to form running stitches. Turning now to FIG. 17, an exemplary running stitch 35 over a tissue or wound interface 32 can be formed by inserting the disclosed suture 10 over and along a first portion 36 of the interface 32, intersecting the suture 10 to form an interlocking structure 33 (with optional tying of one or more knots over the interlocking structure 33), and, without breaking the suture 10, inserting the suture 10 over and along a second portion 37 of the interface 32. As illustrated in FIG. 17, a plurality of the interlocking structures 33 may be formed along the length of the interface 32 to effectively reduce or break up the tension between the tissue and the running stitch 35.

Again, in some procedures, especially when optimal scar appearance is important, it would be desirable to form the running stitches beneath the surface of the wound or tissue interface 32. To that end, FIG. 18 illustrates a subcutaneous or subcuticular running stitch 38 that can be formed by the disclosed suture 10. Specifically, the subcutaneous or subcuticular running stitch 38 can be formed by inserting the disclosed suture 10 subcutaneously (e.g. zigzag) along a first portion 39 of the interface 32, intersecting the suture 10 subcutaneously to form an interlocking structure 33 (with optional tying of one or more subcutaneous knots over the interlocking structure 33), and, without cutting the suture 10, inserting the suture 10 subcutaneously along a second portion 40 of the interface 32. Unlike traditional suture, the running subcuticular sutures 38 may involve areas of intersection 33 that effectively “lock” the segment of running stitches to provide enhanced stitch security and/or to prevent transmission of tension induced deformities (e.g. “bunching”) along the entire length of the running stitch. The surgeon may choose to put these areas of intersection (which effectively form an anchoring knot where the suture is looped upon itself) at an area where the surgeon wishes to lock the segment of closure. However, these areas of intersection do not need to be placed at every portion of the running stitches, just where the locking effect is desired. For example, the ability to lock segments of running stitches may be desired when areas of tissue excess along one aspect of a wound interface need to be “gathered in” or tailored to achieve an even wound closure.

As explained earlier in the present disclosure, and without wishing to be bound by any particular theory, one feature of the present disclosure is that the shape of the projections 13, the orientation and arrangement of the projections 13 around the core 11, the cross-sectional profile of the core 11, the shape of the slots 22, and combinations thereof, may allow the suture 10 of the present disclosure to form interlocking intersections to improve suture security and/or knot security.

Moreover, the suture 10 according to the present disclosure achieves knot security by incorporating structural features to serve particular functions, rather than by putting more throws in a knot as in the conventional sutures. As a result, the suture of the present disclosure may reduce or minimize mass of foreign material in the tissue or wound interface, thereby reducing the risk of inflammation and scarring, as well as the occurrence of knot spitting associated with conventional sutures. In addition, reducing the number of knot throws would reduce the time needed to fasten each stitch, thereby reducing overall operative time, particularly for longer wound interfaces where more stitches are required.

Finally, the security of the suture according to the present disclosure is significantly enhanced by the interlocking structure formed at the suture intersection and therefore does not rely entirely on the engagement between the suture and the tissue. As a result, the suture of the present disclosure may have improved holding strength than barbed sutures. Moreover, because the suture of the present disclosure does not need to run back and forth across the tissue interface (e.g. wound or incision) to increase the length of the tissue engagement for better security, the tissue interface may have less tension-induced deformation (e.g. serpentine or sinusoidal configurations) compared to barbed sutures. For example, while a barbed suture may cause tissue to bunch up along the length of a wound closure to form a serpentine wound interface, especially when pulled too tight, the suture of the present disclosure can be locked and/or looped at various intervals along the wound closure so that the “bunching” effect and the serpentine deformation can be prevented or at least reduced.

While only certain embodiments have been set forth, alternative embodiments and various modifications will be apparent from the above descriptions to those skilled in the art. These and other alternatives are considered equivalents and within the spirit and scope of this disclosure.

What is claimed is:

1. A surgical suture, comprising:
an elongated core extending along an axis; and
at least one axial array of projections provided on the core, each projection extending from the core and terminating into an outer edge, the outer edge of the projection having an axial length greater than a remainder of the projection.

2. The surgical suture of claim 1, wherein each projection further comprises two opposing sidewalls extending from the core and terminating into the outer edge.

3. The surgical suture of claim 2, wherein the two opposing sidewalls are at least substantially parallel to each other.

4. The surgical suture of claim 2, wherein the sidewalls are planar.

5. The surgical suture of claim 2, wherein the sidewalls are non-planar.

6. The surgical suture of claim 1, wherein the outer edge of the projection is parallel to the axis.

7. The surgical suture of claim 1, wherein the outer edge of the projection is non-parallel to the axis.

8. The surgical suture of claim 7, wherein the outer edge of the projection comprises two interconnected sections positioned at different angles relative to the axis.

9. The surgical suture of claim 1, wherein the suture comprises two axial arrays of projections extending in opposite directions.

10. The surgical suture of claim 1, wherein the suture comprises more than two axial arrays of projections evenly spaced around the core.

11. A surgical suture, comprising:
an elongated core extending along an axis; and
at least one axial array of projections extending from the core, each two adjacent projections defining a slot, each slot including an opening having an axial length smaller than a remainder of the slot.

12. The surgical suture of claim 11, wherein each projection terminates into an outer edge.

13. The surgical suture of claim 11, wherein the projections are planar.

14. The surgical suture of claim 11, wherein the projections are non-planar.

15. The surgical suture of claim 11, wherein the suture comprises two axial arrays of projections extending in opposite directions.

16. The surgical suture of claim 11, wherein the suture comprises more than two axial arrays of projections evenly spaced around the core.

17. A method for forming a surgical stitch, the method comprising:
providing a surgical suture having an elongated core extending along an axis and at least one axial array of projections extending from the core; and
intersecting the surgical suture to form at least one loop, the intersection being secured by at least one of projection-projection engagement and projection-core engagement.

18. The method of claim 17, further comprising tying at least one knot over the loop.

19. The method of claim of 17, wherein each projection terminates into an outer edge, the outer edge of the projection being axially wider than the remainder of the projection.

20. The method claim of 17, wherein each two adjacent projections defines a slot and each slot has a radial opening axially narrower than the remainder of the slot.
